(19) United States
(12) Patent Application Publication
(10) Pub. No.: US 2005/0028234 A1
(43) Pub. Date: Feb. 3, 2005

(54) Transgenic plants over-expressing C-repeat/dehydration-responsive element-binding factor

(76) Inventors: Ming-Tsair Chan, Taipei (TW); Yee-Yung Charng, Taipei (TW); Ning-Sun Yang, Taipei (TW); Than-Hua David Ho, Chesterfield, MO (US)

Correspondence Address: FISH & RICHARDSON PC, 225 FRANKLIN ST, BOSTON, MA, 02110, US

(21) Appl. No.: 10/630,365

(22) Filed: Jul. 30, 2003

(51) Int. Cl. A01H 1/00; C12N 15/82; C12Q 1/68; C12N 5/04

(52) U.S. Cl. 800/288; 435/419

(57) ABSTRACT

A transformed plant cell or transgenic plant containing a recombinant nucleic acid that encodes a heterologous C-repeat/dehydration-responsive element-binding factor (CBF). Expression of the CBF factor increases tolerance of the cell or the plant to chilling, oxidative stress, water-deficit, or salt. Also disclosed are methods of producing such a transformed plant cell or transgenic plant.

### BACKGROUND

Water deficit, high salinity, and chilling are important environmental factors that cause osmotic stress and adverse effects on growth of plants and productivity of crops. Production of water deficit-, chilling-, and salt-tolerant transgenic crops is critical for agriculture. Genetic engineering has been used as a relatively fast, precise, and cost-effective means of achieving improved stress-tolerance in certain plants (McNeil et al. (1999) Plant Physiol. 120:945-950).

### SUMMARY

This invention relates to expressing a heterologous C-repeat/dehydration-responsive element-binding factor (CBF) in a plant cell that is naturally chilling-sensitive, thereby increases tolerance of the cell to chilling, oxidative stress, water-deficit, or salt.

In one aspect, the invention features a transformed plant cell containing a recombinant nucleic acid that encodes a heterologous CBF factor. Expression of the CBF factor increases tolerance of the cell to chilling, oxidative stress, water-deficit, or salt. The CBF factors useful for this invention include, for example, Arabidopsis CBF1. The expression of the CBF factor can be driven, e.g., by a stress-inducible promoter. The plant cell can be a dicot plant cell (e.g., a tomato cell) that is naturally chilling-sensitive.

A transformed plant cell of this invention can be cultivated to generate a transgenic plant. Such a transgenic plant is within the scope of the invention. More specifically, the invention features a transgenic plant whose genome contains a recombinant nucleic acid encoding a heterologous CBF factor. Expression of the CBF factor increases tolerance of the plant to chilling, oxidative stress, water-deficit, or salt.

A “recombinant nucleic acid” is a nucleic acid the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein.

A “naturally chilling-sensitive” cell or plant, as used herein, refers to a cell or plant that does not cold-acclimate under normal growth conditions.

In another aspect, the invention features a method of producing a transformed plant cell. The method involves introducing into a plant cell a recombinant nucleic acid that encodes a heterologous CBF factor and expressing the CBF factor in the cell to increases tolerance of the cell to chilling, oxidative stress, water-deficit, or salt.

Also within the scope of this invention is a method of producing a transgenic plant. The method involves introducing into a plant cell a recombinant nucleic acid encoding a heterologous CBF factor, expressing the CBF factor in the cell, and cultivating the cell to generate a plant. Expression of the CBF factor increases tolerance of the plant to chilling, oxidative stress, water-deficit, or salt. When the plant is treated with an exogenous gibberellic acid (e.g., GA₃), the plant does not exhibit the dwarf phenotype.

The details of some embodiments of the invention are set forth in the description below. Other features, objects, and advantages of the invention will be apparent from the description, and from the claims.

### DETAILED DESCRIPTION

This invention is based on an unexpected discovery that expression of Arabidopsis CBF1 in tomato increases tolerance of the plant to chilling, oxidative stress, water-deficit, or salt.

Accordingly, the invention features a transformed plant cell containing a recombinant nucleic acid that encodes a heterologous CBF factor. Expression of the CBF factor, e.g., driven by a stress-inducible promoter, increases tolerance of the cell to chilling, oxidative stress, water-deficit, or salt. The CBF factors useful for this invention include, for example, Arabidopsis CBF1, CBF2, and CBF3. The plant cell can be a dicot plant cell, e.g., a tomato cell, a brassica cell, or a potato cell, or a monocot plant cell, e.g. a rice cell, a wheat cell, or a barley cell.

A transformed plant cell of the invention can be produced by introducing into a plant cell a recombinant nucleic acid that encodes a heterologous CBF factor and expressing the CBF factor in the cell to increases tolerance of the plant to chilling, oxidative stress, water-deficit, or salt.

Techniques for transforming a wide variety of plant cells are well known in the art and described in the technical and scientific literature. See, for example, Weising et al. (1988) Ann. Rev. Genet. 22:421-477. To express a heterologous CBF factor gene in a plant cell, the gene can be combined with transcriptional and translational initiation regulatory sequences which will direct the transcription of the gene and translation of the encoded protein in the plant cell.

For example, for overexpression, a constitutive plant promoter may be employed. A “constitutive” promoter is active under most environmental conditions and states of cell differentiation. Examples of constitutive promoters include the cauliflower mosaic virus (CaMV) 35S transcription initiation region, the 1′- or 2′-promoter derived from T-DNA of Agrobacterium tumafaciens, the ACT 11 and Cat3 promoters from Arabidopsis (Huang et al. (1996) Plant Mol. Biol. 33:125-139 and Zhong et al. (1996) Mol. Gen. Genet. 251:196-203), the stearoyl-acyl carrier protein desaturase gene promoter from Brassica napus (Solocombe et al. (1994) Plant Physiol. 104:1167-1176), the GPc 1 and Gpc2 promoters from maize (Martinez et al. (1989) J. Mol. Biol. 208:551-565 and Manjunath et al. (1997) Plant Mol. Biol. 33:97-112).

Alternatively, a plant promoter may be employed to direct expression of the CBF factor gene in a specific cell type (i.e., tissue-specific promoters) or under more precise environmental or developmental control (i.e., inducible promoters). Examples of environmental conditions that may affect transcription by inducible promoters include anaerobic conditions, elevated temperature, the presence of light, spray with chemicals or hormones, or infection of a pathogen. Examples of such promoters include the root-specific ANRI promoter (Zhang and Forde (1998) Science 279:407) and the photosynthetic organ-specific RBCS promoter (Khoudi et al. (1997) Gene 197:343).

For proper polypeptide expression, a polyadenylation region at the 3′-end of the coding region should be included. The polyadenylation region can be derived from the natural gene, from a variety of other plant genes, or from T-DNA.

A marker gene can also be included to confer a selectable phenotype on plant cells. For example, the marker gene may encode a protein that confers biocide resistance, antibiotic resistance (e.g., resistance to kanamycin, G418, bleomycin, hygromycin), or herbicide resistance (e.g., resistance to chlorosulfuron or Basta).

A recombinant nucleic acid that encodes a heterologous CBF factor may be introduced into the genome of a desired plant host cell by a variety of conventional techniques. For example, the recombinant nucleic acid may be introduced directly into the genomic DNA of a plant cell using techniques such as electroporation and microinjection of plant cell protoplasts, or the recombinant nucleic acid can be introduced directly to plant tissue using ballistic methods, such as DNA particle bombardment.

Microinjection techniques are known in the art and well described in the scientific and patent literature. The introduction of a recombinant nucleic acid using polyethylene glycol precipitation is described in Paszkowski et al. (1984) EMBO J. 3:2717-2722. Electroporation techniques are described in Fromm et al. (1985) Proc. Natl. Acad. Sci. USA 82:5824. Ballistic transformation techniques are described in Klein et al. (1987) Nature 327:70-73.

Alternatively, the recombinant nucleic acid may be combined with suitable T-DNA flanking regions and introduced into a conventional Agrobacterium tumefaciens host vector. The virulence functions of the Agrobacterium tumefaciens host will direct the insertion of the CBF factor gene and adjacent marker into the plant cell DNA when the cell is infected by the bacteria. Agrobacterium tumefaciens-mediated transformation techniques, including disarming and use of binary vectors, are well described in the scientific literature. See, for example, Horsch et al. (1984) Science 233:496-498; Fraley et al. (1983) Proc. Natl. Acad. Sci. USA 80:4803; and Gene Transfer to Plants, Potrykus, ed., Springer-Verlag, Berlin, 1995.

The presence and copy number of the heterologous CBF factor gene in a transgenic plant can be determined using methods well known in the art, e.g., Southern blotting analysis. Expression of the heterologous CBF factor gene in a transgenic plant may be confirmed by detecting the heterologous CBF factor mRNA or protein in the transgenic plant. Means for detecting and quantifying mRNA or proteins are well known in the art.

Transformed plant cells which are derived by any of the above transformation techniques can be cultured to regenerate a whole plant. Such regeneration techniques rely on manipulation of certain phytohormones in a tissue culture growth medium, typically relying on a biocide or herbicide marker that has been introduced together with the heat shock factor gene. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. (1987) Ann. Rev. Plant Phys. 38:467-486. Once the heterologous CBF factor gene has been confirmed to be stably incorporated in the genome of a transgenic plant, it can be introduced into other plants by sexual crossing. Any of a number of standard breeding techniques can be used, depending upon the species to be crossed.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

### EXAMPLE 1

### Heterologous Expression of the Arabidopsis CBF1 Gene Confers Elevated Tolerance to Chilling and Oxidative Stresses in Transgenic Tomato

### Materials and Methods

Plant Materials

Arabidopsis thaliana (L.) Hyen. ecotype Columbia was grown in controlled environment chambers at 24° C., 50% relative humidity, with 24 h photoperiod (about 120 μmol m⁻²s⁻¹). Seeds of Lycopersicon esculentum (L.) Miller cv. CL5915-93D₄-1-0-3, kindly provided by Asian Vegetable Research and Development Center (AVRDC), Tainan, Taiwan, were soaked at 32° C. for 1 h and surface sterilized for 10 min with 1% NaOCl, and washed twice with sterile water for 5 min and subsequently germinated on MS medium with a 16 h photoperiod at 26° C.

DNA Construct

A CBF1 gene was isolated by reverse transcriptase polymerase chain reaction (RT-PCR) from 3-week-old Arabidopsis leaves as described previously (Chan and Yu (1998) Plant J. 15:685-695). Two primers covering the whole CBF1 coding region were chosen to amplify a 640 bp DNA fragment. The 5′ primer (5′-ACGCGTCGACATGAACTCATTTTCAGCTTTT3′) and the 3′ primer (5′-CGAGCTCTTAGTAACTCCAAAGCGACA3′) were located at the translation initiation site (ATG) and the stop site (TAA) of the CBF1 coding region, respectively. A pfu DNA polymerase (Promega) was used to amplify the DNA fragment to minimize the chance of sequence mutation. The 640 bp PCR product was cloned into the T7Blue(R) vector (Novagen) to form pT7Blue-CBF1 and the DNA sequence was determined by an ABI PRISM 373 automatic DNA sequencing system. The CBF1 cDNA was then cloned into pJD301 (Luehrsen et al. (1992) Methods in Enzymology 216:397-441) by removing its luciferase gene to form the intermediate vector. The fragment containing a CaMV35S promoter, tobacco mosaic virus (TMV) Ω leader, a CBF1 gene and a nos poly(A) was excised by digesting with BamHI and BglII from the intermediate vector and cloned into the BamHI site of pCAMBIA 2301 (Center for the Application of Molecular Biology of International Agriculture, Australia) to form pJLM1. The pCAMBIA 2301 vector contains two other selectable markers, GUS and NPTII genes driven by two separate 35S promoters. Plasmid was transformed into A. tumefaciens strain LBA4404 cells by electroporation.

Production of Transgenic Tomato Plants

Cotyledons of 7- to 10-day-old tomato seedlings were used for transformation.

Individual cotyledons were crosscut into two pieces and precultured upside down on KCMS medium (Fillatti et al. (1987) Biotech 5:726-730) containing 100 μM acetosyringone (AS) for one day. The subsequent plant transformation procedure was performed as described (Fillatti et al. (1987) Biotech 5:726-730).

Analysis of Transgenic Tomato Plants

To identify positive transgenic lines for Southern analysis, genomic DNA from rooted putative transformants growing on MS medium with 100 mg/l kanamycin sulfate was extracted as described previously (Chan et al. (1993) Plant Mol. Biol. 22:491-506). Total RNA isolation, as well as DNA and Northern blot analyses, was performed as described (Chan et al. (1994) J. Biol. Chem. 269:17635-17641). The β-glucuronidase (GUS) DNA isolated from the BamHI-SacI restriction fragment of plasmid pBI221 (Clontech), and the CBF1 gene isolated from pT7Blue-CBF 1 were used as probes. Tomato β-TUBULIN cDNA fragment was isolated by RT-PCR from 3-month-old tomato plant leaves. The 5′ primer (5′-CCCGGGCACACTTGATCCCATTCGT-3′, SmaI site underlined) and the 3′ primer (5′-CCCGGGCATTCTGTCTGGGTACTCT-3′, SmaI site underlined) were chosen to amplify the 539 bp β-TUBULIN partial cDNA fragment. The PCR fragments were cloned into pT7Blue(R) and the DNA sequences were determined by an ABI PRISM 373 automatic DNA sequencing system. CAT1 (GenBank accession number: M93719) was isolated from subtractive hybridization and excised from pT7Blue(R) vector as probes. These fragments were labeled with [α-³²P]dCTP using the random primer method (Feinberg and Vogelstein (1983) Anal. Biochem. 132:6-13). The GUS histochemical staining assay was performed according to previously described methods (Chan et al. (1993) Plant Mol. Biol. 22:491-506). Tomato seeds produced from transgenic tomato plants were collected and selection procedures performed as described above.

Analysis of Transgenic Plants Under Cold and Oxidative Stress Conditions

To evaluate cold tolerance, the transgenic T₁, and wild-type plants were placed into cold conditions under 16/8 hours light (about 120 μmol m⁻²s⁻¹) for various time (1, 3, 5, and 7 days). Leaves excised from the wild type and the transgenic tomato plants were immersed in deionized water and subjected to ion leakage determination with a conductivity meter. The sample was then autoclaved to destroy the cells and release all ions. The value obtained after autoclaving was designated as 100% electrolyte leakage. Survival rate was defined as the number of healthy plants following incubation at 0° C. for 7 days, and transfer to room temperature for 5 days, divided by the total number of plants treated in this manner. Pictures were also taken to record the phenotypes.

Chlorophyll fluorescence values of paraquat-treated leaf discs and whole leaves of chilling-treated tomato plants were measured using a pulse-activated modulation fluorimeter (Walz, Effeltrich, Germany) according to the method described by Oberschall et al. (2000) Plant J. 24:437-446. The amount of H₂O₂ was assayed as previously described by O'Kane et al. (1996) Planta 198:366-370.

GA₃ and Cold Treatment of Wild-Type and Transgenic Tomato Plants

Wild-type and 3-week-old transgenic T₁, tomato plants were sprayed with 5 ppm GA₃ three times for one week. After GA₃ treatment, whole plants were incubated at 0° C. for 1, 3, 5, and 7 days, then chlorophyll fluorescence was tested. Total chlorophyll in leaf tissue was measured according to previously described methods (Deak et al. (1999) Nature Biotech. 17:192-196).

The T₂ seeds chilling tolerance test of homozygous transgenic lines and wild-type tomato plants was performed basically according to the method of Rab and Sailveit with some modification (Rab and Sailveit (1996) J. Am. Soc. Hort. Sci.121:711-715). T₂ seeds were soaked overnight at 25° C., and transferred to 3 layers of wet paper towel between two 19×19.5-cm glass plates that were oriented according to the protocol. For GA₃ pre-treatment, the transgenic and wild type germinated seeds were soaked in water containing 5 ppm GA₃ at 25° C. for 2 days before cold treatment. The cold treatment was set at 2° C. for 5 days in the dark, and transferred to 25° C. for 3 days under 16/8 hours light (about 120 μmol m⁻²s ⁻¹) for radical regrowth following cold treatment. The subsequent radical elongation was measured and recorded at the end of the 3-day regrowth period.

Protein Extractions, Western Blot Analysis and Enzyme Assays

Whole leaves of the transgenic and wild type tomato plants were used for enzyme extractions and analyses. The protein extraction and activity assay of catalase enzyme were carried out as described by Pinhero et al. (1997) Plant Physiol. 114:695-704. The Western blot analysis was performed as previously described (Chan et al. (1994) J. Biol. Chem. 269:17635-17641). To prepare anti-Arabidopsis CBF1 antibodies, the CBF1 cDNA isolated from pT7Blue-CBF1was subcloned into the pET24b vector containing histidine tag (Novagen), and then transformed into E. coli BL21 (DE3). The expressed recombinant protein was purified with a histidine tag affinity column (Novagen). The overexpression and purification of the CBF1 protein was performed as previously described (Kanaya et al. (1999) J. Biol. Chem. 274:16068-16076). CBF1 polyclonal antibodies were raised in rabbits according to standard procedure.

Subtractive Hybridization

0.7 μg polyA⁺RNA extracted from wild type and transgenic tomato plants were used to perform subtractive hybridization according to the Clontech PCR select cDNA subtraction kit manual (Clontech). Amplified PCR products were cloned into pT7Blue(R) vector (Novagen). Next, DNA sequence was determined by an ABI PRISM 373 automatic DNA sequencing system.

Results

Identification of Transgenic Tomato Plants that Overexpress CBF 1

In order to evaluate the effect of overexpression of CBF in tomato plants, an Arabidopsis CBF1 and the marker gene GUS were transferred into the tomato genome using Agrobacterium-mediated transformation. After selection on kanamycin-containing medium, the putative transgenic tomato plants were further identified by GUS histochemical staining assays to screen for transgenic tomato plants expressing the transgenes. Twenty-two independent lines were obtained and Southern blot analysis showed that 35S:CBF1 transgene was integrated into the genome of the transformed plants. Northern blot analysis was performed to evaluate the 35S:CBF1 transgene expression in three transgenic tomato lines. CBF1 and GUS RNA transcripts were detected only in the transgenic lines, while the amount of β-TUBULIN RNA was similar in the transgenic and wild type tomato plants. These plants were further characterized by Western blot analysis of protein extracts from the leaf tissues with polyclonal antibodies raised against the recombinant CBF 1 protein. The antibodies recognized a protein of c.a. 25 kDa molecular mass in the samples from the transgenic tomato plants, whereas there was no signal in the wild-type plants. These results indicated that the transgene was successfully expressed transcriptionally and translationally in the transgenic plants. However, using Arabidopsis COR47, KIN1, and COR15a cDNA as probes, we did not detect any corresponding COR homologous gene expression in either wild type or transgenic tomato plants even in low stringent hybridization conditions. Neither did we observe any accumulation in transgenic tomato plants of the tomato dehydrin TAS14. Tomato dehydrin TAS14 is responsive to salinity, ABA, and mannitol (Godoy et al. (1990) Plant Mol. Biol. 15:695-705, Godoy et al. (1994) Plant Mol. Biol. 26:1921-1934, and Parra et al. (1996) Plant Mol. Biol. 32: 453-460).

Transgenic Tomatoes Exhibited Enhanced Chilling Tolerance but Not Freezing Tolerance

Due to the fact that overexpression of CBF1 in Arabidopsis confers freezing tolerance, we were interested to know whether CBF1 has a similar effect on transgenic tomato plants. We found that neither transgenic nor wild type plants were able to survive −2° C. for 2 days followed recovery at 24° C. However, the transgenic plants exhibited enhanced chilling tolerance comparing to wild-type plants. Photosynthesis efficiency as measured by light-induced chlorophyll fluorescence (Fv/Fm) and ion leakage, were measured to reflect the level of cellular damage following chilling treatment at various temperature (0° C., 2° C., 4° C., 6° C., or 24° C.) for 7 days. The Fv/Fm ratio decreased in the wild-type plants after 3 days at 2° C. and 0° C.; however, the transgenic plants appeared to be less affected. Cold stress also caused severe ion leakage in the wild-type plants, whereas the transgenic plants were much less affected.

After treatment at 0° C. for 1 day, leaves of the wild-type plants became wilted and curled. However, after recovery at 24° C. from chilling treatment, young leaves of the wild-type plants resumed vigorous growth. To estimate the survival rate after cold treatment, we decide to extend the time of low-temperature treatment (0° C.) to 7 days, and then returned the plants to 24° C. for recovery. The transgenic tomatoes were more tolerant to the chilling treatment without showing severe stress symptoms, while virtually all leaves of the wild-type plants became wilted and curled. All of the wild-type plants eventually died, whereas 83.3%, 80%, and 75% of C5, C15, and C21 plants survived, respectively.

CBF1 Transgenic Tomatoes Exhibited Dwarf Phenotype That Can be Overcome by Exogenous GA₃ Treatment

When comparing the phenotypes of the transgenic tomato plants with those of the wild-type plants, severe growth retardation of the transgenic tomato plants was observed. All transgenic plants were considerably shorter than the wild-type plants. The average height of various transgenic plants was less than 50% of that of the wild-type plant, due to shorter internodes in the transgenic plants. We also observed a significant reduction of the number of fruit and seed set in transgenic plants under normal conditions. Since gibberellin (GA) content has been shown to correlate with internode length (Ross et al. (1989) Physiol. Plant 76:173-176), we were interested to know whether application of GA could overcome the dwarf phenotype of the transgenic plants. Application of GA₃ to the transgenic tomatoes permitted essentially growth to the normal height. These results suggested that the heterologous CBF1 protein might affect the genes for hormone production involved in growth.

Chilling Tolerance of Transgenic Tomato Plants is Not Affected by Exogenous GA₃ Treatment

After GA₃ treatment, plants were incubated at 0° C. for various time (1, 3, 5, and 7 days), ion leakage and Fv/Fm value were measured. After recovery from chilling (0° C.), the percent leakage of ions and Fv/Fm value were less affected in transgenic tomato plants, despite GA₃ treatment, than wild type. There was no discernible effect of GA₃ treatment on any of the stress-resistance parameters tested, regardless of genotype. The results showed that chilling tolerance of transgenic tomato plants was not affected by application of exogenous GA₃. Moreover, after recovered from 5 days storage at 2° C., the radical growth of wild-type seedlings was significantly reduced comparing to those without cold treatment. However, the T₂ CBF 1 transgenic seedlings were able to ameliorate the growth inhibition resulting from chilling treatment with or without GA₃ pre-treatment. These results also showed that chilling tolerance of transgenic tomato T₂ seedlings was not affected by application of exogenous GA₃.

Constitutive Activation of Catalase Gene in Transgenic Tomatoes

Overexpression of CBF1 can induce expression of COR genes in Arabidopsis (Jaglo-ottosen et al. (1998) Science 280:104-106). To see whether overexpression of CBF1 could induce the expression of COR homologues in tomato, we have performed Northern blot analysis of the transgenic plants by employing Arabidopsis COR genes as probes. RNA transcripts from transgenic tomato plants did not cross hybridize to the Arabidopsis COR47, KIN1, and CORI 15a even in low stringent hybridization conditions. These results suggested that the increased tolerance against chilling stress may not be due to the expression of known COR gene homologues in transgenic tomato. To determine which tomato genes might be induced by CBF1 protein, a subtractive hybridization experiment was performed. We isolated several cDNAs that accumulated either to a greater or lesser amount in the transgenic plants compared to the wild type. The identities of these cDNA clones were revealed by BLAST search of the GenBank database. No known COR homologues were among the cDNAs we isolated, while the CATALASE1 (GenBank accession number: M93719) gene transcript was the most prominent. Northern blot analysis using the tomato CAT 1 cDNA as a probe showed that the level of CAT 1 RNA transcripts was about two-fold greater in the three transgenic tomato lines than in wild-type plants in non-stressed conditions. Arabidopsis CBF1 was only expressed in the 3 transgenic tomato lines and transgene expression did not perturb the expression of β-TUBULIN. These results indicated that the expression of CBF 1 in transgenic plants influenced the expression of CAT1 gene either directly or indirectly.

Expression of the CAT1 Gene and Catalase Activity were Maintained at High Levels in Chilling-Stressed Transgenic Tomato Plants

Northern blot was performed to examine the response of tomato CAT1 toward low temperature treatment. The amount of CAT1 transcript was significantly reduced in both the wild type and transgenic plants, but remained high in transgenic plants under chilling-stress. We also measured the level of CAT activity in crude leaf extracts, from the transgenic and wild type plants under normal- and chilling-conditions. The results were consistent with those of Northern blot analysis of CAT 1 transcripts. CAT activities decreased after chilling stress both in the transgenic and wild type plants; however, the enzyme activities in the transgenic plants were still higher than that of the wild-type plants under the same condition. Because CAT activity was affected, we also determined the H₂O₂ content in the transgenic tomato plants. The amount of H₂O₂ in the transgenic plants was significantly lower than that in the wild-type plants under normal- or chilling-conditions. The mRNA expression patterns of CAT1 gene were similar in the three transgenic tomato plant lines under chilling stress.

Enhanced Tolerance to Oxidative Stress in Transgenic Tomatoes

Because the catalase activity and H₂O₂ content in the transgenic plants were altered, we investigated the response of transgenic plants to paraquat, an oxidative stress-inducing agent. Paraquat treatment caused about 50% reduction in the Fv/Fm value in the wild-type plants, whereas the transgenic plants suffered a lesser loss. The damage caused by paraquat was also reflected by the degree of bleaching of the tissues. The total chlorophyll content decreased up to 60% in the wild type by paraquat treatment, but the loss in the transgenic plants was less than 30%.

### EXAMPLE 2

### Tomato Plants Ectopically Expressing Arabidopsis CBF1 Show Enhanced Tolerant to Water Deficit Stress

### Materials and Methods

Plant Materials

Seeds of Lycopersicon esculentum (L.) Miller cv. CL5915-93D₄-1-0-3, were provided by the Asian Vegetable Research and Development Center. Seeds were soaked in water at 32° C. for 1 hr, surface sterilized for 10 min with 1% NaOCl, washed twice with sterile water for 5 min, and subsequently germinated on MS medium under a 16 hr photoperiod at 26° C.

DNA Construction and Agrobacterium-Mediated Tomato Transformation

A CBF 1 gene was isolated by reverse transcriptase polymerase chain reaction (RT-PCR) from 3-week-old Arabidopsis leaves as described previously (Chan and Yu (1998) Plant J. 15:685-695). The transformation procedure has been described previously (Hsieh et al. (2002) Plant Physiol., in press).

Identification of Transgenic Tomato Plants

All rooted shoots growing on MS medium with 100 mg/l kanamycin sulfate were tested by GUS staining (0.1 M Na-phosphate buffer, pH 7.0, 10 mM EDTA, 0.1% Triton X-100, 1 mM X-Gluc, 0.5 mM K-ferrocyanide, pH 7.0, 0.5 mM K-ferricyanide, pH 7.0) (Chan et al. (1993) Plant Mol. Biol. 22:491-506), and then subjected to Southern and Northern blot analyses. Genomic DNA was extracted from all rooted shoots on MS medium with 100 mg/l kanamycin sulfate as described previously (Chan et al. (1993) Plant Mol. Biol. 22:491-506). Total RNA was isolated using a Triazole (Gibcol) solution, the DNA and RNA blot analyses were performed according to Chan et al. (1994) J Biol. Chem. 269:17635-17641. The β-glucuronidase (GUS) DNA, excised from the BamHI-SacI restriction fragment of plasmid pBI221 (Clontech), and the CBF 1 gene, isolated from pT7Blue-CBF 1, were used as probes. Tomato β-TUBULIN cDNA fragment was isolated by RT-PCR from 3-month-old tomato plant leaves. The 5′ primer (5′-CCCGGGCACACTTGATCCCATTCGT-3′, SmaI site underlined) and the 3′ primer (5′-CCCGGGCACACTTGATCCCATTCGT-3′, Smal site underlined) were chosen to amplify the 539 bp β-TUBULIN partial cDNA fragment. The PCR fragments were cloned into pT7Blue(R) and the DNA sequences were determined by an ABI PRISM 373 automatic DNA sequencing system. CAT1 (GenBank accession number: M93719) isolated from subtractive hybridization was also used as a probe. All fragments were labeled with [α-³²P]dCTP using the random primer method (Feinberg and Vogelstein (1983) Anal. Biochem. 132:6-13). Tomato seeds produced from transgenic tomato plants were collected and selection procedures were performed as described previously.

Analysis of Transgenic Plants Under Water Deficit Conditions

Wild type and transgenic tomato plants were grown in pots with peat moss and watered every other day. Day temperature was maintained at 26° C.±2° C. and night temperature was maintained at 22° C.±2° C. Relative humidity was maintained at 50%±10%. Plants were grown under 16/8 hours light (about 120 μmol m⁻²s⁻¹). Survival rate of plants treated with water deficit was defined as the number of healthy plants divided by the total number of plants. Pictures were taken to record the phenotypes. The water deficit treatment time was included in the growth period. After a total of 3 months, these plants were harvested, weighted for fresh weight, and the numbers of fruits and seeds were calculated. Each analysis was repeated at least five times.

For water deficit treatment, wild type and transgenic T₁, plants were grown at 24° C. and without water supply for various time periods (0, 7, 14, 21, 28 days). For GA₃ treatment, three week-old wild type and transgenic T₁, plants were sprayed with 5 ppm GA₃ three times within a week. Three leaves or five roots were detached from each plant and weighed for fresh weight, with sampling and measurements repeated five times. Detached leaves or roots were then dried at 65 ° C. for 2 days to determine dry weight. The water content (WC) was calculated based on the following equation WC=(fresh weight−dry weight)/(dry weight)=g·H₂O/g·DW. Chlorophyll fluorescence values were measured using a pulse-activated modulation fluorimoter (Walz, Effeltrich, Germany) according to the method described by Oberschall et al. (2000) Plant J. 24:437-446.

Leaf conductance measurements were taken from the third and fourth leaves of intact transgenic CBF1 and wild-type plants in the presence of (control) and absence of water (7 days) (water deficit stress). The results were measured at an interval of a 10 hr light and 2 hr dark. Leaf conductance was measured with a LI-COR LI- 1600 steady state porometer (LI-COR, Lincoln, Nebr., USA).

Proline Content , Catalase Activity and H₂O₂ Concentration Analyses

Leaves detached from plants were extracted using 3 sulfosalicylic acid, and the supernatants collected after centrifugation. The supernatants were incubated with ninhydrin and acetic acid at 100° C. for 60 min, cooled in ice to terminate the reaction. Toluene was then added and the absorbance at A₅₂₀ measured.

Wild type and transgenic T₁, plants were grown at 24 ° C. without water for 28 days as described previously (water deficit treatment). The leaf catalase activity was measured according to Pinhero et al. (1997) Plant Physiol. 114:695-704. The H₂O₂ concentration was analyzed as described in O'Kane et al. (1996) Planta 198:366-370.

Subtractive Hybridization

PolyA⁺RNA (0.7 pg) was extracted from leaves of wild-type and transgenic tomato plants grown under normal conditions, and used to perform subtractive hybridization according to the Clontech PCR select cDNA subtraction kit manual (Clontech). After PCR amplification, the PCR products were cloned into the pT7Blue(R) vector (Novagen). DNA sequences were determined by an ABI PRISM 373 automatic DNA sequencing system.

### Results

Identification of Transgenic Tomato Plants

A DNA cassette consisting of an Arabidopsis CBF1 cDNA driven by a CaMV 35S promoter and terminated with a nos signal was ligated into pCAMBIA2301, which contains GUS and NPTII reporter genes, to form pJLM1. This plasmid was transferred into the tomato genome using Agrobacterium-mediated transformation. After kanamycin selection, the putative transgenic tomato plants were determined by GUS staining to identify the transgenic plants. We obtained 22 unique transgenic lines and performed GUS staining for C5, C15, and C21 lines only. Leaves of transgenic tomato lines C5, C15, and C21 showed blue color and wild-type leaves showed white color. Southern blot analysis revealed that one copy of the heterologous CBF1 gene was integrated into tomato genome of transgenic tomato lines C5 and C15. After digestion with BamHI, a foreign CBF1 DNA was detected as a fragment of the expected size of 12 kb for C5 and 10 kb for C15. Size of the undigested tomato genomic DNA was about 50 kb. After digestion with HindIII, the lower molecular-weight DNA fragments of c.a. 4.7 kb appeared for C5 and C15. Genomic DNA from these plants did not migrate long distance on a 14 cm long 0.8% agarose gel during electrophoresis, which might account for the similarity in the HindIII restriction pattern of transgenes. There is an endogenous CBF1 homolog in the tomato genome, the size of which was found to be 4.7 kb after digestion with BamHI or HindIII.

Overexpression of Heterologous Arabidopsis CBF1 in Transgenic Tomato Plants

Northern blot analysis was performed to reveal the MRNA levels in transgenic T₁, plants. The heterologous CBF1 and GUS transcripts accumulated only in transgenic T₁, plants. Interestingly, one transgenic T₁, plant did not highly express Arabidopsis CBF1; however, the GUS transcripts were present. Transgenic T₁, plants expressing heterologous CBF1 were evaluated for tolerance to water deficit stress. Levels of mRNA of β-TUBULIN and rRNA were used as internal controls.

Transgenic Tomato Plants Showed More Water Deficit Tolerance Than Wild-Type Plants

To evaluate the capacity for water deficit tolerance of transgenic tomatoes, whole plants, grown in the same pot with peat moss, were not watered for 21 days. Leaves of wild-type plants became wilted and curled. In order to examine the survival rates of wild type and transgenic plants under water deficit conditions, the treatment (water deficit) was extended to 4 weeks. The wild-type plants were sick after 28 days without water, and did not recover during the 7-day period following re-watering. Compared to the survival rate of wild-type plants, transgenic tomatoes were apparently more tolerant to water deficit after 4 weeks of water deprivation. Less than 6% of the wild-type plants survived four weeks after water deficit treatment, whereas 77.8%, 83.3%, and 83.3% of transgenic tomato plants C5, C15, and C21 survived, respectively. These results suggest that overexpression of CBF 1 can significantly improve water deficit tolerance in tomato, similar to the results obtained from transgenic Arabidopsis plants overexpressing DREB1A (CBF3) (Kasuga et al. (1999) Nature Biotech. 17:287-291).

The tolerance to water deficit stress was revealed by chlorophyll fluorescence Fv/Fm values, measured at days 0, 7, 14, and 28 during water deficit treatment. Photosystem II integrity was significantly more stable in transgenic plants compared to the wild-type plants during water stress. Fv/Fm decreased in wild-type plants after 21 days without water and did not recover after re-watering. The transgenic plants, however, maintained an Fv/Fm value at about 50% of the normal value even after 28 days without water, and recovered almost completely after re-watering.

In order to test the ability of maintaining water in the tissue, water content of leaves and roots of water deficit-stressed transgenic tomato and wild-type plants were measured at various time points. Water content of transgenic plants remained high during water deficit treatment. In contrast, a marked reduction in water content was observed in wild-type plants.

Water Deficit Tolerance Not Affected by Applying GA₃, in Transgenic Tomato Plants

All transgenic tomato plants were shorter than wild-type plants due to shorter internodes. Previously, internode length was reported to positively correlate with GA content (Ross et al. (1989) Physiol. Plant 76:173-176). Recently, we found that the internode length of transgenic plants could be recovered by applying GA₃ (gibberellic acid) exogenously (Hsieh et al. (2002) Plant Physiol., in press). It was of interest to know now whether the tolerance to water deficit stress of transgenic tomato plants would be affected by GA₃ treatment. GA₃-treated wild type and transgenic tomato plants were subjected to water deficit stress as previously described. Fv/Fm values and the water content of leaves and roots showed that transgenic tomato plants were still more tolerant to water deficit stress than GA₃-treated wild-type plants. It is also worth noting that GA₃ treatment of both wild type and transgenic tomato plants seems to have little or no impact on water deficit tolerance. GA content seems to correlate with internode length, but water deficit tolerance seems to be independent of GA content. Hence, the ability to tolerate water deficit stress was not affected by GA₃ in transgenic tomato plants. These results suggested that the CBF 1-mediated improvement of water deficit tolerance in transgenic tomato plants was probably not due to morphological changes.

To identify the possible relationship between stomatal movement and water imbalance, changes of leaf conductance were determined. Leaf conductance in the wild-type plants followed a typical diurnal pattern. In all transgenic tomato and wild-type plants, the stomatal opening increased rapidly after the start of the light period, reached a maximal level at about six hours, and then decreased. The stomata of the transgenic CBF1 tomato plants closed rapidly after water deficit treatment with or without GA₃ pre-treatment compared to wild-type plants, which showed a similar pattern to that under regular watering. CBF1 expression apparently resulted in retained water thus negating tissue damage, and therefore, the phenotype of transgenic plants appeared normal under water deficit conditions.

More Proline Was Detected in Transgenic Tomato Plants

Many plants respond to water deficit by accumulating high concentrations of compatible solutes or osmolytes, such as proline, mannitol, fructose, glycine betaine and trehalose (Bajaj et al. (1999) Mol. Breeding 5:493-503 and Hoekstra et al. (2001) Trends Plant Sci. 6:1360-1385). Because elevated proline levels occurs in transgenic Arabidopsis that overexpressed DREB1A (CBF3) (Gilmour et al. (2000) Plant Physiol. 124:1854-1865), we also measured proline content in transgenic tomato plants under normal and water deficit conditions. The proline content in transgenic tomato was higher than in wild-type plants under both normal and water deficit conditions. GA₃ treatment did not affect the proline content in wild type or transgenic tomato plants. These results suggest that, consistent with the higher Fv/Fm value and water content, transgenic tomato plants possess an inherent tolerance of water deficit, which is much better than wild-type plants.

Exogenous GA₃ Treatment Reversed Growth Retardation of Transgenic Tomato Plants Without Affecting Water Deficit Tolerance

Transgenic tomato plants were not only shorter than wild-type plants, but also had fruit and seed numbers and fresh weight less than those of wild-type plants under normal growth conditions. After treatment with exogenous GA₃, fruit and seed numbers and fresh weight increased, suggesting that GA₃ improved the growth retardation of transgenic tomato plants, although the seed numbers were not the same as for GA₃-treated wild-type plants. After water deficit treatment, the fresh weight of transgenic tomato plants was more than wild-type plants, which died after the treatment. No difference in fruit and seed numbers of transgenic tomato plants under normal or water deficit conditions was observed. In contrast, the production and fresh weight of wild-type plants were severely impaired by water deficit treatment, indicating that they were less tolerant to water deficit comparing to transgenic tomato plants. Exogenous GA₃ treatment showed the same results as for non-GA₃ treatment, suggesting that water deficit tolerance was not affected by treatment with exogenous GA₃ in transgenic tomato plants, which was consistent with no changes in fruit and seed numbers and fresh weight. Because the phenomenon of chilling treatment was similar to water deficit treatment, we also calculated the fruit and seed numbers and fresh weight after chilling treatment. Results of chilling treatment were similar to water deficit treatment, suggesting that the transgenic tomato plants were more tolerant to chilling- and water deficit-stress than wild-type plants.

Enhancement of CAT1 Expression, Catalase Activity, and Reduction of H₂O₂ Concentration in Transgenic Tomato Plants

Transgenic tomato plants were analyzed at the molecular level using subtractive hybridization techniques against wild-type plants. Subtractive hybridization experiments were performed in order to identify any up-regulated genes in transgenic tomato plants. No known COR homologous genes were isolated from the subtractive hybridization experiment, and Arabidopsis COR genes, such as COR47, KIN1, and COR15a did not cross-hybridize with any tomato RNA even under low stringency hybridization conditions. Using tomato dehydrin TAS14, which is also responsive to stresses (Godoy et al. (1990) Plant Mol. Biol. 15:695-705, Godoy et al. (1994) Plant Mol. Biol. 26:1921-1934, and Parra et al. (1996) Plant Mol. Biol. 32:453-460) as a probe, we failed to detect any mRNA transcripts expressed in any transgenic tomato plants. The CAT1 gene, however, was one of a number of up-regulated genes we isolated. Northern blot analysis indicated that transcriptional levels of CAT1 were higher in transgenic tomatoes than in wild-type plants under normal or water deficit conditions. The catalase and H₂O₂ concentrations of plants grown under normal conditions or without watering for 28 days were measured. Catalase activity of transgenic tomato plants was higher than that of wild-type plants under normal or water deficit conditions. H₂O₂ concentrations were lower in transgenic tomato than in wild-type plants under normal or stress conditions. Our results indicate that CAT 1 expression and catalase activity increased and H₂O₂ concentration was reduced in transgenic tomato plants.

### EXAMPLE 3

### Activated Oxidative-Responsive Genes in CBF1 Transgenic Tomatoes Enhance Salt Tolerance

### Materials and Methods

Plant Materials

Seeds of Lycopersicon esculentum (L.) Miller cv. CL5915-93D₄-1-0-3, were provided by Asian Vegetable Research and Development Center. Seeds were soaked in water at 32° C. for 1 hr, sterilized for 10 min with 1% NaOCl, washed twice with sterile water for 5 min, and subsequently germinated on MS medium with a 16 hr photoperiod (about 120 μmol m⁻²s⁻¹) at 26° C.

Plasmid DNA Construction and Agrobacterium-Mediated Tomato Transformation

Isolation of Arabidopsis CBF1 full-length cDNA has been described previously (Hsieh et al. (2002) Plant Physiol., in press and Hsieh et al. (2002) Plant Physiol. 129:1086-1094). The DNA cassette containing a CaMV35S promoter, tobacco mosaic virus (TMV) Ω leader, CBF1 cDNA and a nos poly(A) tail was excised with BamHI and BglII and cloned into the BamHI site of pCAMBIA2301 (Center for the Application of Molecular Biology of International Agriculture, Australia) to form pJLM 1. pJLM 1 was then transformed into A. tumefaciens strain LBA4404 cells by electroporation. Cotyledon were cut from 7- to 10-day-old tomato seedlings and used for Agrobacterium-mediated transformation, as described previously (Hsieh et al. (2002) Plant Physiol., in press and Hsieh et al. (2002) Plant Physiol. 129:1086-1094).

Identification of Transgenic Tomato Plants

All rooted shoots on MS medium with 100 mg/l kanamycin sulfate were tested by GUS histochemical staining (0.1 M Na-phosphate buffer, pH 7.0, 10 mM EDTA, 0.1% Triton X-100, 1 mM X-Gluc, 0.5 mM K-ferrocyanide pH 7.0, 0.5 mM K-ferricyanide, pH 7.0) (Chan et al. (1993) Plant Mol. Biol. 22:491-506), and then subjected to Southern and Northern blot analyses as described previously (Chan et al. (1993) Plant Mol. Biol. 22:491-506 and Chan et al. (1994) J. Biol. Chem. 269:17635-17641). Probes used were as fallows: the β-glucuronidase (GUS) cDNA fragment was excised from plasmid pBI221 (Clontech) at BamHI-SacI sites and the CBF1 gene isolated from pT7Blue-CBF1 (Hsieh et al. (2002) Plant Physiol. 129:1086-1094) at SalI-SacI sites; tomato β-TUBULIN, CAT1 (GenBank accession number: M93719) and GST-like (GenBank accession number: AY082341) cDNA fragments were excised from pT7Blue-β-TUBULIN at Smal sites and pT7Blue-CAT1 at RsaI sites. These cDNA fragments were labeled with [α-³²P]dCTP using the random primer method (Feinberg and Vogelstein (1983) Anal. Biochem. 132:6-13). Tomato seeds produced from transgenic tomato plants were collected and the selection procedures were performed as described above.

Salt Treatment

Two-month-old plants were grown in Hogland's solution (Johnson et al. (1957) Plant and Soil 8:337-353) at 24° C.±2 ° C. under 16/8 hours light (about 120 μmol m⁻²s⁻¹) with Hogland's solution changed every 7 days. For salt treatment, wild type and transgenic plants were incubated in Hogland's solution with 200 mM NaCl. Photographs were taken and chlorophyll fluorescence values were measured after salt treatment using a pulse-activated modulation fluorimoter (Walz, Effeltrich, Germany) according to the method described by Oberschall et al. (2000) Plant J. 24:437-446.

For survival rate tests, wild type and transgenic T₂ plants were grown in the same pot and watered with 200 mM NaCl regularly for 3 months. At the end of treatment, fruit and seed numbers of surviving plants and the total fresh weight of plants were calculated.

For catalase activity analysis, wild type and transgenic tomatoes were incubated in Hogland's solution with 200 mM NaCl for 3 days. Leaves were then collected and measured for catalase activity according to Pinhero (1997) Plant Physiol. 114:695-704. For ascorbate peroxidase, glutathione reductase and peroxidase activity analyses, wild type and transgenic tomatoes were incubated with 140 mM NaCl for 3 days. Leaves were then collected and measured for enzyme activities as described previously (Foster and Hess (1980) Plant Physiol. 66:482-487, MacAdam et al. (1992) Plant Physiol. 1992: 872-878, Nakano and Asada (1981) Plant Cell Physiol. 22:867-880, and Pinhero et al. (1997) Plant Physiol. 114:695-704).

### Results

Plasmid Construction and Identification of Transgenic Tomato Plants

Arabidopsis CBF1 cDNA driven by a CaMV 35S promoter and terminated using a nos signal was ligated into pCAMBIA2301, which contains GUS and NPTII marker genes, to form pJLM1. This plasmid was then transferred into the tomato genome using an Agrobacterium-mediated transformation system. After kanamycin selection, putative transgenic tomato plants were confirmed by GUS staining. We obtained 22 different T₀ transgenic lines but have only shown indicative GUS staining for C5, C15 and C21 transgenic lines. Transgenic tomato C5, C15 and C21 lines had blue leaves whilst wild-type leaves were white, indicative that the GUS gene was only expressed in transgenic plants. Genomic DNA extracted from wild type and transgenic plants was digested with BamHI and HindIII for Southern blot analysis using Arabidopsis CBF1 cDNA as a probe. In wild-type plants, the heterologous CBF1 cross-hybridized a band of c.a. 4.5 kb after BamHI digestion and a band of c.a. 9.4 kb after HindIII digestion, indicating presence of an endogenous CBF1 homolog in the tomato genome. An extra band observed in transgenic tomato lines C5, C15, and C21 revealed that one copy of the heterologous CBF1 gene was integrated into the tomato genome of transgenic tomato lines C5, C15, and C21.

The transcriptional level of heterologous CBF1 in transgenic T₁, plants was determined using CBF1 and GUS cDNA, as probes to hybridize the total RNA extracted from T₁, plants derived from the 3 T₀ lines C5, C15 and C21. Heterologous CBF1 and GUS reporter genes were only expressed in transgenic T₁, plants, not in wild-type plants grown under normal conditions. β-TUBULIN and rRNA were used as loading controls. These T₁, plants were used to evaluate salt tolerance of transgenic plants in comparison to wild-type plants.

CBF1 Transgenic Tomato Plants Showed Salt Tolerance

Previously, we showed that overexpression of heterologous CBF1 could enhance chilling-, and oxidative-stress tolerance and water deficit resistance in transgenic tomato plants (Hsieh et al. (2002) Plant Physiol., in press and Hsieh et al. (2002) Plant Physiol. 129:1086-1094). We now determined whether overexpressed heterologous CBF1 could improve tolerance of transgenic tomato plants to salt stress. The salt tolerance of transgenic tomato or wild-type plants was evaluated by incubating the plants in 200 mM NaCl in the same water jar for 3 days. Wild-type leaves curled and wilted; however, transgenic plants did not exhibit such symptoms. Wild type and transgenic T₂ plants were then grown in the same pot and watered with 200 mM NaCl every second day for 3 months in order to determine survival rates. The survival rates of wild type and transgenic T₁, plants were 0%, and 70%, 83.3%, 66.6% for C5, C15, and C21 lines, i.e., more transgenic plants survived 3 months of salt treatment than wild-type plants.

The Fv/Fm values of transgenic tomato plants were also determined by measuring chlorophyll fluorescence. Fv/Fm values in wild-type plants decreased dramatically after salt treatment. However, the Fv/Fm values did not significantly change in transgenic plants in the presence or absence of salt, suggestive that photosystem II integrity was more stable in transgenic plants than in wild-type plants. These results indicated that transgenic tomato plants were more tolerant to salt stress than wild-type plants.

Salt Tolerance of Transgenic Tomato Plants Was Not Affected by Exogenous GA₃ Treatment

Previously, we reported that growth retardation exhibited by transgenic tomato plants overexpressing CBF 1 could be reversed by exogenous GA₃ treatment without affecting chilling tolerance and water deficit resistance (Hsieh et al. (2002) Plant Physiol., in press and Hsieh et al. (2002) Plant Physiol. 129:1086-1094). We now examined the effect of GA₃ on salt tolerance of transgenic tomato plants. Growth retardation of plants was calculated by noting fruit and seed numbers and the fresh weight of plants at the end of salt treatment. The results showed that the fruit and seed numbers and fresh weight of wild-type plants not treated with GA₃ decreased, whilst there was no significant difference observed in transgenic T₂ plants not treated with GA₃, corroborating previous evidence that transgenic plants were more tolerant to salt stress than wild-type plants. The fruit and seed numbers and fresh weight of GA₃ treated wild-type plants decreased under saline conditions. Exogenous GA₃ treated transgenic plants had their growth retardation reversed (Hsieh et al. (2002) Plant Physiol., in press); however, after salt treatment, there was no significant change of fruit and seed numbers and fresh weight in transgenic T₂ plants, suggestive that salt tolerance was not influenced by GA₃ treatment. These results showed that transgenic tomato plants exhibited salt tolerance regardless of GA₃ treatment.

Increase of CAT1, Ascorbate Peroxidase, Glutathione Reductase and Peroxidase Activities in Transgenic Tomato Plants

CAT1 gene expression is constitutively activated in transgenic tomato plants under normal and chilling conditions (Hsieh et al. (2002) Plant Physiol. 129:1086-1094). Hence, we determined whether CAT1 transcription was also up-regulated in transgenic tomato plants under salt stress. After incubation of wild type and transgenic plants at a concentration in Hogland's solution with 200 mM NaCl for 3 days, expression of CAT1 gene was up-regulated in wild-type plants and three different transgenic lines C5, C15, C21. CAT1 transcripts were more abundant in transgenic tomatoes than in wild-type plants under both stressful and non-stressful conditions. A GST homolog was isolated by subtractive hybridization with CAT1 (Hsieh et al. (2002) Plant Physiol. 129:1086-1094) and named GST-like gene (GenBank accession number: AY082341). The deduced amino acid sequence showed 82% identity with tomato GST (glutathione S-transferase/peroxidase; GenBank accession number: AAF22647). Results of Northern blot analysis using a partial cDNA fragment of the GST-like gene as a probe showed down-regulation of the GST-like gene after salt treatment in wild-type and three different transgenic lines C5, C15, C21; however, more transcripts were detected in transgenic tomato plants than in wild-type plants with or without salt treatment. Catalase, ascorbate peroxidase, glutathione reductase, and peroxidase activities also showed up-regulation after salt treatment both in wild type and transgenic tomatoes; however, these anti-oxidant enzyme activities were higher in transgenic tomatoes than in wild-type plants. Therefore, these results revealed that overexpression of heterologous CBF1 in transgenic tomato plants are associated with increased activation of anti-oxidant enzymes.

### EXAMPLE 4

### Expression of Arabidopsis CBF1 Regulated by ABA/stress Inducible Promoter in Transgenic Tomato Confers Stress Tolerance without Affecting Yield

### Material and Methods

Plant Materials

Arabidopsis thaliana L. Hyen. ecotype Columbia was grown in controlled environment chamber at 24° C., 50% relative humidity, with 24 hr photoperiod (about 120 μmol m−2s−1). The seeds of Lycopersicon esculentum L. Miller cv. CL5915-93D4-1-0-3, kindly provided by Asia Vegetable Research and Development Center (AVRDC), were soaked in room temperature for 1 hr, surface sterilized for 15 min with 1% NaOCI, washed twice with sterile water for 5 min, and subsequently germinated on MS medium with 16hr photoperiod at 26° C.

Plasmid Construction

A CBF1 gene was isolated by reverse transcriptase polymerase chain reaction (RT-PCR) from 3 weeks old Arabidopsis leaves as described previously (Hsieh et al. (2002) Plant Physiol. 129:1086-1094). The CBF1 cDNA was then cloned into pJD301 (Luehrsen et al. (1992) Methods Enzymol 216:397-414) by removing the luciferase gene to form the CBF1/pJD301 intermediate vector. The ABRC3 promoter, three copies of ABA-response complex (ABRC1) from barley HV A22 fused to a minimal promoter, truncated (−60) barley α-amylase (Am64) promoter, was digested with NotI and NcoI from pQS122 (Shen and Ho (1995) Plant Cell 7:295-307). This promoter fragment was filled in with Klenow and ligated to the HindIII and SalI sites of CBF1/pJD301. The ABRC1/CBF1/nos fragment was digested with EcoRI, and ligated to the EcoRI site of pCAMBIA 2301 to form pJLM2. The plasmid was transformed to Agrobacterium LBA4404 by electroporation.

Tomato Transformation

The tomato transformation procedure was carried out according to the method described in Hsieh et al. (2002) Plant Physiol. 129:1086-1094.

Analysis of Transgenic Tomato Plants

Total RNA isolation, as well as DNA and RNA blot analysis, was performed as described (Chan et al. (1994) J. Biol. Chem. 269:17635-17641). Total RNA isolated from T₂ plants was used for Northern blot analysis. The CBF1 gene isolated from pT7Blue-CBF1 was used as a probe (Hsieh et al. (2002) Plant Physiol. 129:1086-1094). The CAT1 (GenBank accession number: M93719) was isolated using subtractive hybridization and excised from pT7Blue(R) vector as a probe (Hsieh et al. (2002) Plant Physiol. 129:1086-1094). These fragments were labeled with [α-³²P]dCTP using the random primer method (Feinberg and Vogelstein (1983) Anal. Biochem. 132:6-13).

Analysis of Transgenic Plants Under Chilling, Water Deficit, and Salt Stress Conditions

To evaluate chilling tolerance, two months old transgenic T₂ and wild-type plants were incubated at 0C. under 16/8 hr light (about 120 μmol m⁻²s⁻¹) for 7 days, then transferred to room temperature for further growth. To identify the water deficit tolerance, two months old wild type and transgenic T₂ plants were incubated at 26° C. without water supply for 4 weeks. For salt tolerance experiment, two months old wild type and transgenic T₂ plants were watered with 200 mM NaCl for 4 weeks. The nutrient solution was Hogland's solution, which was replaced every six days. Except for the stress treatment period, all plants were maintained under similar growth conditions for further analysis. Day temperature was maintained at 26° C.±2° C. and night temperature was maintained at 22° C.±2° C. Relative humidity was maintained at 50% +10%. Plants were grown under 16/8 hours light (about 120 μmol m⁻²s⁻¹). All tomato plants were regularly watered every 2 days and fertilized with stock fertilizer 4 times during the growth period. The survival rate of plants was defined as the number of healthy plants divided by the total number of plants. Pictures were also taken to record the phenotypes. The stress treatment time was also included in the growth period. After a total of 3 months, the plants were harvested and weighted for fresh weight, and the number of fruits and seeds were calculated. For ion leakage determination, leaves excised from the wild type and transgenic tomato plants were immersed in deionized water and subjected to the ion leakage determination with a conductivity meter. For leaf conductance measurement, the third and fourth leaves of intact transgenic CBF1 and wild-type plants with water and incubated at 24° C. (control), with water and incubated at 0° C. (chilling stress) for 2 days, without water for 10 days (water deficit stress), or treated with 200 mM NaCl for 4 days (salt stress) were used for the determination of leaf conductance. After various treatments, the results were obtained under 10 hr light and 2 hr dark period. Leaf conductance was measured with a LI-COR LI-1600 steady state porometer (LI-COR, Lincoln, Nebr., USA). The sample was then autoclaved to destroy the cells and release all ions. The value obtained after autoclaving was designated as 100% electrolyte leakage. Chlorophyll fluorescence values of whole leaves of various stress-treated tomato plants were measured using a pulse-activated modulation fluorimeter (Walz, Effeltrich, Germany) according to the method described by Oberschall et al. (2000) Plant J. 24:437-446. Each analysis was repeated at least ten times.

### Results

Expression of Transgene CBF1 in Response to ABA, Chilling, Water Deficit and Salt Treatment in Transgenic Tomatoes

To determine whether ABRC1 cis-acting element of barley HV A22 gene could be activated and drive CBF 1 to regulate downstream genes in tomato plants, transgenic tomato plants were treated with chilling, water deficit and salt, respectively. Total RNA was prepared from leaves of transgenic plants after various treatments. The CBF1 gene is not regulated by ABA or dehydration in Arabidopsis (Medina et al. (1999) Plant Physiol. 119:463-470). It was also observed that no CBF 1 regulation is present in wild-type tomato treated with various stress conditions or ABA. Contrarily, the CBF1 transcript driven by ABRC I promoter could be induced in transgenic tomatoes under water deficit and salt treatments while no signal was observed without the treatments. However, the CBF1 transcript was barely induced by chilling. These results indicated that expression of CBF1 could be induced by various stress treatments in transgenic ABRC-CBF1 tomato plants.

It was known that tomato catalase1 (CAT 1) gene could be induced by heterologous CBF1 protein (Hsieh et al. (2002) Plant Physiol. 129:1086-1094). The expression level of CBF1 in individual transgenic tomato plants correlated with the concomitant transactivation of the CAT1 gene. The higher expression of CBF1 observed, the higher activation of CAT1. Furthermore, the expression level of CAT1 was similar in wild type and transgenic plants because no CBF 1 transcripts were detected in transgenic plants under non-treatment conditions. These results indicated again that the CAT1 gene is responsive to CBF1 directly or indirectly.

Reversibility of Expression of ABRC1-CBF1 in Transgenic Tomatoes

To identify whether ABRC1 promoter could be reversible under normal growth conditions and environmental stress, transgenic tomato was alternatively grown with and without water for various times. Leaves were collected at various times, total RNA was extracted, and CBF1 transcripts were detected by Northern blot analysis. CBF1 transcripts were undetectable in transgenic tomato grown under regular water conditions while significantly increased after water deficit treatment. When the water deficit-treated plants were returned to regular water conditions, the CBF1 transcripts appeared rapidly. The amount of CBF1 transcripts increased again after transgenic tomato was treated with water deficit stress. We could still detect a few CBF 1 transcripts when water deficit-treated transgenic tomatoes were returned to regular water conditions. No CBF1 transcript was detected in wild-type plants even after water deficit treatment. These results demonstrated that the activation or inhibition of ABRC1-CBF 1 in transgenic tomato was reversible and mediated by the presence or absence of environmental stress.

Transgenic ABRC1-CBF1 Tomatoes with Enhanced Tolerance to Chilling, Water Deficit , and Salt Stresses

Previously, overexpression of Arabidopsis CBF1 in tomato plants was found to result in tolerance to chilling stresses (Hsieh et al. (2002) Plant Physiol. 129:1086-1094). We therefore tried to determine the stress-tolerance ability of these transgenic lines in planta. Wild type and transgenic tomato plants were tested for stress tolerance. Wild-type tomato plants seemed sensitive to various stress conditions, while transgenic ABRC-CBF1 lines showed tolerance to chilling, water deficit, and salt. All of the wild-type plants eventually died, whereas about 83.3%, 93.3%, and 96.7% of AC1, AC2 and AC3 plants survived under water deficit and salt stresses, respectively. In view of the Northern data, both water deficit and salt stress highly induced CBF1 expression in transgenic lines, which were tolerance to these stresses. But when chilling treatment was tested, cold injuries such as wilting, chlorosis, and necrosis appeared in leaves of both wild type and transgenic plants. During chilling stress period, the difference of chilling damage on both wild-type and transgenic plants did not differ significantly. However, when the chilling-treated plants were returned to normal growth conditions, the transgenic plants recovered significantly faster than the control plants. Moreover, almost none of the wild-type plants survived, whereas about 65%, 50%, and 55% of AC1, AC2, and AC3 plants recovered, respectively.

Photosynthesis efficiency as measured by light-induced chlorophyll fluorescence (Fv/Fm) and ion leakage were measured to reflect the level of cellular damage following various stress treatments. The Fv/Fm ratio decreased in the wild-type plants under various stress conditions, while the transgenic plants appeared to be less affected. Various stresses also caused severe ion leakage in the wild-type plants, whereas the transgenic plants were much less affected.

Yields of Transgenic ABRC1-CBF1 Tomatoes Were Not Affected by Environmental Stresses

Previously, we observed that transgenic tomato plants overexpressing CBF 1 are shorter than wild-type plants due to shorter internodes (Hsieh et al. (2002) Plant Physiol. 129:1086-1094). However, the transgenic ABRC1-CBF1 tomato plants did not exhibit any notable morphological abnormalities such as growth retardation. The fruit number, seed number, and fresh weight of transgenic tomato were the same as for the wild-type plants under normal growth conditions. Moreover, the fruit number was higher for transgenic tomato overexpressing CBF1, the C5 plant, with or without GA₃ treatment. After various osmotic treatments, the fresh weight of transgenic tomato was more than that of wild-type tomato which died after the treatment. Furthermore, no significant difference in fruit and seed numbers of transgenic tomato plants under normal or various osmotic conditions was observed. In contrast, the production of wild-type plants was severely impaired after various osmotic treatments, indicating that wild-type plants were not tolerant to various osmotic treatments compared to transgenic tomato plants. These results indicate that transgenic plants maintain similar yield under various osmotic conditions as compared to wild-type plants under normal growth conditions.

Transgenic Plants Closed Stomata Rapidly Under Various Stresses

To identify the possible relationship between stomatal movement and water imbalance, changes of leaf conductance were determined. Leaf conductance in the wild-type plants showed a standard diurnal change. In all transgenic tomato and wild-type plants, the stomatal movement increased rapidly after the start of the light period, reached a maximal level at about the sixth hour, and then decreased. The stomatal movement of the transgenic tomato plants decreased rapidly after various stress treatments for about 2 h. In contrast, the leaf conductance of wild-type plants undergoing various stresses started to close more slowly than transgenic plants at about 6 h. These results implied that the transgenic plants showed more sensitivity to various stress treatments. Furthermore, expression of CBF1 apparently resulted in retained water thus negated tissue damage. The phenotype of transgenic plants was normal when exposed to various osmotic stresses.

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

1. A transformed plant cell comprising a recombinant nucleic acid that encodes a heterologous C-repeat/dehydration-responsive element-binding factor (CBF), wherein the cell is naturally chilling-sensitive and expression of CBF increases tolerance of the cell to chilling, oxidative stress, water-deficit, or salt.

2. The transformed plant cell of claim 1, wherein the factor is Arabidopsis CBF1.

3. The transformed plant cell of claim 2, wherein the expression of the factor is driven by a stress-inducible promoter.

4. The transformed plant cell of claim 3, wherein the transformed plant cell is a dicot plant cell.

5. The transformed plant cell of claim 4, wherein the transformed plant cell is a tomato cell.

6. The transformed plant cell of claim 2, wherein the transformed plant cell is a dicot plant cell.

7. The transformed plant cell of claim 6, wherein the transformed plant cell is a tomato cell.

8. The transformed plant cell of claim 1, wherein the expression of the factor is driven by a stress-inducible promoter.

9. The transformed plant cell of claim 8, wherein the transformed plant cell is a dicot plant cell.

10. The transformed plant cell of claim 9, wherein the transformed plant cell is a tomato cell.

11. The transformed plant cell of claim 1, wherein the transformed plant cell is a dicot plant cell.

12. The transformed plant cell of claim 11, wherein the transformed plant cell is a tomato cell.

13. A transgenic plant comprising a recombinant nucleic acid that encodes a heterologous C-repeat/dehydration-responsive element-binding factor (CBF), wherein the plant is naturally chilling-sensitive and expression of the factor increases tolerance of the plant to chilling, oxidative stress, water-deficit, or salt.

14. The transgenic plant of claim 13, wherein the factor is Arabidopsis CBF1.

15. The transgenic plant of claim 14, wherein the expression of the factor is driven by a stress-inducible promoter.

16. The transgenic plant of claim 15, wherein the transgenic plant is a dicot plant.

17. The transgenic plant of claim 16, wherein the transgenic plant is tomato.

18. The transgenic plant of claim 14, wherein the transgenic plant is a dicot plant.

19. The transgenic plant of claim 18, wherein the transgenic plant is tomato.

20. The transgenic plant of claim 13, wherein the expression of the factor is driven by a stress-inducible promoter.

21. The transgenic plant of claim 20, wherein the transgenic plant is a dicot plant.

22. The transgenic plant of claim 21, wherein the transgenic plant is tomato.

23. The transgenic plant of claim 13, wherein the transgenic plant is a dicot plant.

24. The transgenic plant of claim 23, wherein the transgenic plant is tomato.

25. A method of producing a transformed plant cell, the method comprising:
introducing into a plant cell a recombinant nucleic acid encoding a heterologous C-repeat/dehydration-responsive element-binding factor (CBF), and
expressing the factor in the cell,
wherein the cell is naturally chilling-sensitive and expression of the factor increases tolerance of the cell to chilling, oxidative stress, water-deficit, or salt.

26. The method of claim 25, wherein the factor is Arabidopsis CBF1.

27. The method of claim 25, wherein the expression of the factor is driven by a stress-inducible promoter.

28. The method of claim 25, wherein the plant cell is a dicot plant cell.

29. The method of claim 28, wherein the plant cell is a tomato cell.

30. A method of producing a transgenic plant, the method comprising:
introducing into a plant cell a recombinant nucleic acid encoding a heterologous C-repeat/dehydration-responsive element-binding factor (CBF),
expressing the factor in the cell, and
cultivating the cell to generate a plant,
wherein the plant is naturally chilling-sensitive and expression of the factor increases tolerance of the plant to chilling, oxidative stress, water-deficit, or salt.

31. The method of claim 30, wherein the factor is Arabidopsis CBF1.

32. The method of claim 30, wherein the expression of the factor is driven by a stress-inducible promoter.

33. The method of claim 30, further comprising growing the plant in the presence of an exogenous gibberellic acid.

34. The method of claim 30, wherein the transgenic plant is a dicot plant.

35. The method of claim 34, wherein the transgenic plant is tomato.


SEQUENCE LISTING (4 sequences)

SEQ ID NO: 1 - DNA, 31 bp, Artificial Sequence
  Primer
acgcgtcgacatgaactcattttcagctttt

SEQ ID NO: 2 - DNA, 27 bp, Artificial Sequence
  Primer
cgagctcttagtaactccaaagcgaca

SEQ ID NO: 3 - DNA, 25 bp, Artificial Sequence
  Primer
cccgggcacacttgatcccattcgt

SEQ ID NO: 4 - DNA, 25 bp, Artificial Sequence
  Primer
cccgggcattctgtctgggtactct